(19) United States
(12) Patent Application Publication
(10) Pub. No.: US 2019/0365455 A1
(43) Pub. Date: Dec. 5, 2019

(54) TREATMENT INSTRUMENT

(71) Applicant: OLYMPUS CORPORATION, Tokyo (JP)

(72) Inventor: Tsunetaka AKAGANE, Hachioji-shi (JP)

(73) Assignee: OLYMPUS CORPORATION, Tokyo (JP)

(21) Appl. No.: 16/540,109

(22) Filed: Aug. 14, 2019

Related U.S. Application Data
(63) Continuation of application No. PCT/JP2017/005859, filed on Feb. 17, 2017.

(51) Int. Cl. A61B 18/08; A61B 18/14

(52) U.S. Cl. CPC A61B 18/085; A61B 2018/00095; A61B 18/1445

(57) ABSTRACT

A treatment instrument includes a heat conductor which includes a treatment surface and an installation surface, a heat generator which generates heat, a substrate which includes a substrate front surface on which the heat generator is formed and a substrate side surface facing one side in a width direction, the substrate being attached to the installation surface, an adhesive layer which is provided between the installation surface of the heat conductor and the substrate, the adhesive layer being formed of a material having a thermal conductivity and being in close contact with the installation surface, and an insulating portion which is formed of a material having electrical insulation, the insulating portion being in close contact with the heat generator, the substrate front surface, and the substrate side surface.

### CROSS-REFERENCE TO RELATED APPLICATIONS

This is a Continuation Application of PCT Application No. PCT/JP2017/005859, filed Feb. 17, 2017, the entire contents of which are incorporated herein by reference.

### BACKGROUND

U.S. Patent Application Publication No. 2016/0324566 A1 discloses a treatment instrument in which a portion between a pair of grasping pieces is openable or closeable. In this treatment instrument, one grasping piece includes a heat conduction member (heat conductor) having an electrical conductivity and a thermal conductivity, and the heat conduction member includes a treatment surface facing the other grasping piece. In the heat conduction member, a substrate is attached to an installation surface facing a side opposite to the treatment surface, via an adhesive sheet (adhesive layer) having electrical insulation and a thermal conductivity. In addition, in the substrate, a heating element (heating wire) which generates heat with electrical energy supplied is provided on a substrate front surface, and the substrate is attached to the heat conduction member in a state where a side on which the heat conduction member is located faces the substrate front surface. The heat generated by the heating element is transmitted to a treatment surface via the adhesive sheet and the heat conduction member, and is applied from the treatment surface to a treatment target grasped between the pair of grasping pieces. In addition, a conductive member is provided in the other grasping piece, the electrical energy is supplied to the heat conduction member of the one grasping piece and the conductive member of the other grasping piece, and thus, a high frequency current flows to a portion between the heat conduction member and the conductive member through the grasped treatment target.

In the treatment instrument as in U.S. Patent Application Publication No. 2016/0324566 A1, a gap is formed between the substrate and the adhesive sheet. Discharge from a side surface of the substrate to the heat conduction member through this gap may occur. In a case where the discharge occurs between the heating element and the heat conduction member, a voltage resistance of the adhesive sheet is affected.

### BRIEF SUMMARY

According an exemplary embodiment, a treatment instrument includes a heat conductor which includes a treatment surface and an installation surface facing a side opposite to the treatment surface, the heat conductor has a thermal conductivity, a heat generator which generates heat when electrical energy is supplied to the heat generator, a substrate which includes a substrate front surface on which the heat generator is formed and a substrate side surface facing one side in a width direction, the substrate being attached to the installation surface of the heat conductor in a state where the substrate front surface faces a side on which the heat conductor is located, an adhesive layer which is provided between the installation surface of the heat conductor and the substrate, the adhesive layer being formed of a material having a thermal conductivity and being in close contact with the installation surface of the heat conductor, and an insulating portion which is formed of a material having electrical insulation, the insulating portion being in close contact with the heat generator, the substrate front surface, and the substrate side surface.

Advantages will be set forth in the description which follows, and in part will be obvious from the description, or may be learned by practice of the invention. The advantages may be realized and obtained by means of the instrumentalities and combinations particularly pointed out hereinafter.

### BRIEF DESCRIPTION OF THE DRAWINGS

The accompanying drawings, which are incorporated in and constitute a part of the specification, illustrate exemplary embodiments, and together with the general description given above and the detailed description of the embodiments given below, serve to explain the principles of the exemplary embodiments.

FIG. 1 is a schematic view showing a system in which a treatment instrument according to an exemplary embodiment is used.

FIG. 2 is a view schematically showing a cross section substantially perpendicular in a width direction of an end effector according to an exemplary embodiment.

FIG. 3 is a view schematically showing a cross section substantially perpendicular to a longitudinal axis of the end effector according to an exemplary embodiment.

FIG. 4 is a view schematically showing, in a cross section substantially perpendicular to the longitudinal axis, an aspect in which an adhesive layer (adhesive sheet) is disposed around a substrate in a manufacture of the treatment instrument according to an exemplary embodiment.

FIG. 5 is a view schematically showing, in a cross section substantially perpendicular to the longitudinal axis, a state in which the adhesive layer (adhesive sheet) is disposed around the substrate in the manufacture of the treatment instrument according to an exemplary embodiment.

FIG. 6 is a view schematically showing, in a cross section substantially perpendicular to the longitudinal axis, a state in which an adhesive layer (adhesive sheet) is disposed around a substrate in a manufacture of a treatment instrument according to an exemplary embodiment.

FIG. 7 is a view schematically showing, in a cross section substantially perpendicular to the longitudinal axis, an aspect in which an adhesive layer (tube) is formed around a substrate in a manufacture of a treatment instrument according to an exemplary embodiment.

FIG. 8 is a view schematically showing a cross section substantially perpendicular to a longitudinal axis of one grasping piece according to an exemplary embodiment.

FIG. 9 is a view schematically showing a cross section substantially perpendicular to a longitudinal axis of one grasping piece according to an exemplary embodiment.

FIG. 10 is a view schematically showing a cross section substantially perpendicular to a longitudinal axis of one grasping piece according to an exemplary embodiment.

### DETAILED DESCRIPTION

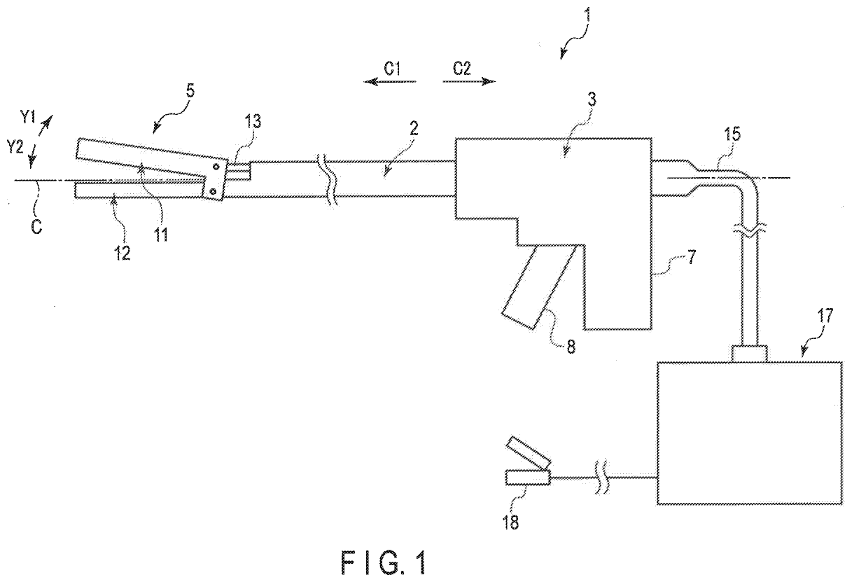

FIG. 1 is a schematic view showing a system in which a treatment instrument 1 according to an exemplary embodiment is used. As shown in FIG. 1, the treatment instrument 1 includes a shaft 2, a housing 3, and an end effector (grasping unit) 5. The shaft 2 has a longitudinal axis C as a central axis and extends along the longitudinal axis C. Here, one side in a direction along the longitudinal axis C is a distal side (arrow C1 side), and a side opposite to the distal side is a proximal side (arrow C2 side). The housing 3 is connected to a proximal side of the shaft 2. In addition, the end effector 5 is provided in a distal portion of the shaft 2.

The housing 3 includes a grip 7 extending along a direction intersecting the longitudinal axis C, and a handle 8 is rotatably attached to the housing 3. As the handle 8 rotates with respect to the housing 3, the handle 8 opens or closes with respect to the grip 7. In addition, in the embodiment of FIG. 1, the handle 8 is located on a side on which the grip 7 is located with respect to the longitudinal axis C, and is located on the distal side with respect to the grip 7. In addition, in each of an opening movement and a closing movement of the handle 8, the handle 8 moves to be substantially parallel to the longitudinal axis C. However, in an embodiment, the handle 8 may be located on the proximal side with respect to the grip 7. In addition, in another embodiment, the handle 8 is located on a side opposite to the side on which the grip 7 is located with respect to the longitudinal axis C, and the handle 8 moves in a direction intersecting (substantially perpendicular to) the longitudinal axis C in the opening movement and the closing movement of the handle 8. In addition, in still another embodiment, an operation member (not shown) such as a rotation knob is attached to the housing 3, the rotation knob rotates around the longitudinal axis C, and thus, the shaft 2 and the end effector 5 rotate together around the longitudinal axis C with respect to the housing 3.

The end effector 5 includes a pair of grasping pieces (jaws) 11 and 12. Here, in an embodiment, one of the grasping pieces 11 and 12 is integrally formed with the shaft 2 or is fixed to the shaft 2, and the other of the grasping pieces 11 and 12 is rotatably attached to the shaft 2. For example, in the embodiment shown in FIG. 1, the grasping piece 11 is rotatably attached to the shaft 2, and the grasping piece 12 is fixed to the shaft 2. In addition, in another embodiment, both the grasping pieces 11 and 12 are rotatably attached to the shaft 2. A movable member 13 extends from the proximal side toward the distal side inside the shaft 2 and a distal portion of the movable member 13 is connected to the end effector 5. In addition, a proximal portion of the movable member 13 is connected to the handle 8 inside the housing 3. The handle 8 is opened or closed with respect to the grip 7, and thus, the movable member 13 moves along the longitudinal axis C. Accordingly, at least one of the grasping pieces 11 and 12 rotates with respect to the shaft 2, and thus, the grasping pieces 11 and 12 is opened or closed. The grasping pieces 11 and 12 can be opened or closed, and thus, a treatment target such as a living tissue can be grasped between the grasping pieces 11 and 12. In addition, a movement direction (directions shown by an arrow Y1 and an arrow Y2) in each of the opening movement and the closing movement of the end effector 5 intersects (substantially perpendicular to) the direction along the longitudinal axis C.

One end of a cable 15 is connected to the housing 3 of the treatment instrument 1. The other end of the cable 15 is connected to an energy source apparatus 17 separated from the treatment instrument 1. In addition, an operation member 18 is provided in the system in which the treatment instrument 1 is used. In the embodiment of FIG. 1, the operation member 18 is a foot switch separated from the treatment instrument 1, and is electrically connected to the energy source apparatus 17. Based on an operation performed by the operation member 18, the energy source apparatus 17 supplies electrical energy to the treatment instrument 1. The electric energy is supplied from the energy source apparatus 17 to the treatment instrument 1, and thus, as described later, treatment energy is applied to the treatment target grasped between the grasping pieces 11 and 12. In addition, in an embodiment, an operation button or the like attached to the housing 3 is provided as the operation member 18 instead of the foot switch or in addition to the foot switch.

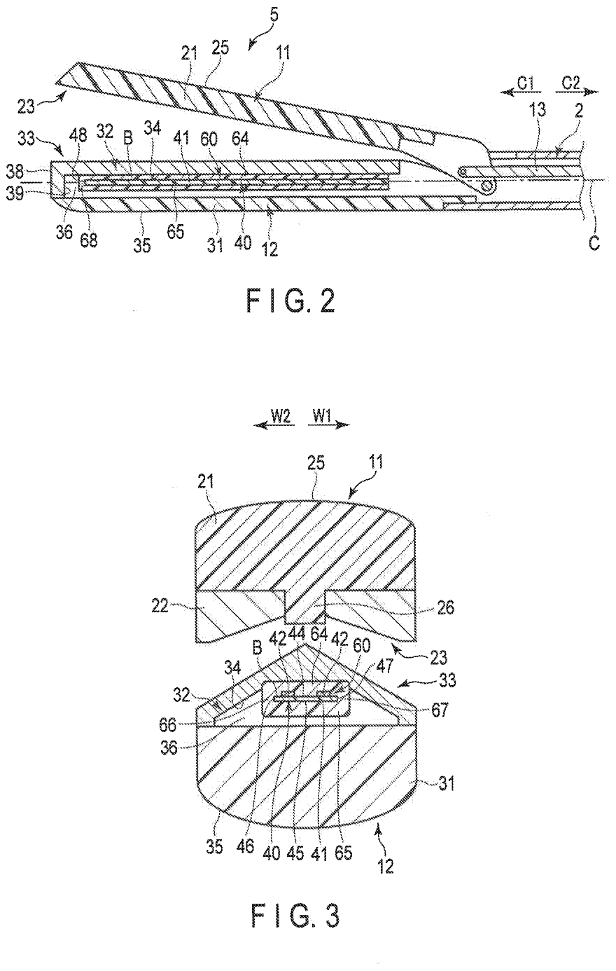

FIGS. 2 and 3 are views showing a configuration of the end effector 5. Here, a width direction (direction shown by an arrow W1 and an arrow W2) of the end effector 5 is defined, which intersects (substantially perpendicular to) the direction along the longitudinal axis C and intersects (substantially perpendicular to) the movement direction in each of the opening movement and the closing movement of the end effector 5. FIG. 2 shows the end effector 5 in a cross section substantially perpendicular to the width direction, and FIG. 3 shows the end effector 5 in a cross section substantially perpendicular to the direction along the longitudinal axis C.

As shown in FIGS. 2 and 3, the grasping piece 11 includes a support 21 which is attached to the shaft 2 and a conductive member 22 which is fixed to the support 21. The conductive member 22 is formed of a metal or the like having an electrical conductivity and is attached to the support 21 from a side on which the grasping piece 12 is located. Each of the support 21 and the conductive member 22 extends over a range from a proximal portion of the grasping piece 11 to a distal portion thereof in the direction along the longitudinal axis C. In addition, the grasping piece 11 includes a facing surface 23 which faces the grasping piece 12 and a rear surface 25 which faces a side opposite to the facing surface 23. In the present embodiment, the rear surface 25 is formed by the support 21, and the facing surface 23 is formed by the support 21 and the conductive member 22.

The support 21 includes a protrusion 26 which protrudes toward the side on which the grasping piece 12 is located, and the protrusion 26 forms a portion of the facing surface 23. The conductive member 22 is provided on both sides of the protrusion 26 in the width direction (width direction of the grasping piece 11) of the end effector 5. In addition, one end of an electric supply path (not shown) formed of an electric wire or the like is connected to the conductive member 22. The electric supply path extends through an inside of the shaft 2, an inside of the housing 3, and an inside of the cable 15, and the other end of the electric supply path is connected to the energy source apparatus 17. In addition, in the support 21, at least a portion being in contact with the conductive member 22 and at least a portion forming the facing surface 23 are formed of a material having electrical insulation. Accordingly, the support 21 is electrically insulated from the conductive member 22. In the embodiment of FIGS. 2 and 3, the entire support 21 including the protrusion 26 is formed of an electrical insulating material. In addition, it is preferable that the support 21 be formed of a material having a low thermal conductivity.

The grasping piece 12 includes a support 31 which is attached to the shaft 2 and a heat conduction member (blade) 32 which is fixed to the support 31. The heat conduction member (heat conductor) 32 is formed of a material having a high thermal conductivity such as a copper alloy or an aluminum alloy and has an electrical conductivity. For example, a thermal conductivity of the heat conduction member 32 is 100 to 500 W/m·K.

In addition, the heat conduction member 32 is attached to the support 31 from the side on which the grasping piece 11 is located. The support 31 and the heat conduction member 32 extend over a range from a proximal portion of the grasping piece 12 to a distal portion thereof in the direction along the longitudinal axis C. In addition, the grasping piece 12 includes a treatment surface (facing surface) 33 which faces the facing surface 23 of the grasping piece 11 and a rear surface 35 which faces a side opposite to the treatment surface 33. In the present embodiment, the rear surface 35 is formed by the support 31 and the treatment surface 33 is formed by the heat conduction member 32.

Moreover, a cavity 36 surrounded by the heat conduction member 32 and the support 31 is formed inside the grasping piece 12. The cavity 36 is formed over a range from the proximal portion of the grasping piece 12 to the distal portion thereof in the direction along the longitudinal axis C. The heat conduction member 32 is adjacent to the cavity 36 from the distal side, a side on which the treatment surface 33 is located, and both sides in the width direction of the end effector 5. In addition, the support 31 is adjacent to the cavity 36 from a side on which the rear surface 35 is located.

One end of the electric supply path (not shown) formed of an electric wire or the like is connected to the heat conduction member 32. The electric supply path extends through an inside of the shaft 2, an inside of the housing 3, and an inside of a cable 15 and the other end of the electric supply path is connected to the energy source apparatus 17. Moreover, in the support 31, at least a portion which is in contact with the heat conduction member 32 and at least a portion adjacent to the cavity 36 are formed of an electrical insulation material. Accordingly, the support 31 is electrically insulated from the heat conduction member 32. In the embodiment of FIGS. 2 and 3, the entire support 31 is formed of an electrical insulation material. In addition, it is preferable that the support 31 be formed of a material having a low thermal conductivity.

The energy source apparatus 17 outputs high frequency electric power as electrical energy based on an operation by the operation member 18. The output high frequency electric power is supplied to the conductive member 22 of the grasping piece 11 via the above-described electric supply path, and the heat conduction member 32 of the grasping piece 12 via the above-described electric supply path. Accordingly, the conductive member 22 and the heat conduction member 32 function as electrodes having different potentials with respect to each other. The conductive member 22 and the heat conduction member 32 function as the electrodes in a state where the treatment target is grasped between the grasping pieces 11 and 12, and thus, the high frequency current flows between the conductive member 22 and the heat conduction member 32 through the treatment target, and the high frequency current is applied to the treatment target as treatment energy.

In addition, in a state where the grasping pieces 11 and 12 is closed, the heat conduction member 32 can abut against the protrusion 26 of the support 21 on the facing surface 23 of the grasping piece 11. In the state where the heat conduction member 32 abuts against the protrusion 26 of the support 21, a gap is formed between the heat conduction member 32 and the conductive member 22, and the heat conduction member 32 does not come into contact with the conductive member 22. Therefore, in the state where the conductive member 22 and the heat conduction member 32 function as the electrodes, a short circuit in an electric circuit of the electrical energy output from the energy source apparatus 17 to the heat conduction member 32 and the conductive member 22 is effectively prevented.

Moreover, in the embodiment of FIGS. 2 and 3, the facing surface 23 of the grasping piece 11 is formed in a concave shape whose central portion in the width direction is recessed toward the rear surface 25, and the treatment surface 33 of the grasping piece 12 is formed in a convex shape whose central portion in the width direction protrudes toward the grasping piece 11 side. Note that, in an embodiment, the facing surface 23 of the grasping piece 11 is provided to be substantially parallel to the width direction of the end effector 5. Moreover, in another embodiment, the facing surface 23 of the grasping piece 11 is formed in a convex shape whose central portion in the width direction protrudes toward the grasping piece 12, and the treatment surface 33 of the grasping piece 12 is formed in a concave shape whose central portion in the width direction is recessed toward the rear surface 35 side.

A heat-generating module (sheet heater) 40 is disposed in the cavity 36 of the grasping piece 12. The heat-generating module 40 includes a substrate 41 and heating elements (heat generator) 42 which are provided on the substrate 41. Each of the substrate 41 and the heating elements 42 extends over a range from the proximal portion of the grasping piece 12 to the distal portion thereof in the direction along the longitudinal axis C. The substrate 41 has electrical insulation. For example, the substrate 41 is a flexible substrate formed of a resin such as polyimide. The heating element 42 has an electrical conductivity. The heating element 42 is a heating wire attached to the substrate 41, a heat-generation pattern printed on the substrate 41, or the like, and is formed of a nichrome alloy, a stainless steel alloy, or the like, for example. The electrical energy is supplied to the heating element 42, and thus, the heating element 42 generates heat caused by a resistance of the heating element 42.

The heat conduction member 32 has an installation surface 34 facing a side opposite to the treatment surface 33. The installation surface 34 is adjacent to the cavity 36 from the side where the treatment surface 33 is located. The heat-generating module 40 is attached to the installation surface 34 via an adhesive layer 60 described later.

The substrate 41 includes a substrate front surface 44 on which the heating elements 42 are formed, and a substrate rear surface 45 which faces a side opposite to the substrate front surface 44. The substrate front surface 44 faces one side in a thickness direction of the substrate 41, and the substrate rear surface 45 faces the other side in the thickness direction of the substrate 41. The heat-generating module 40 is attached to the installation surface 34 of the heat conduction member 32 in a state where the substrate front surface 44 of the substrate 41 faces the side on which the heat conduction member 32 is located. In this case, the thickness direction of the substrate 41 is substantially parallel to the movement direction in each of the opening movement and closing movement of the end effector 5, and the width direction of the substrate 41 is substantially parallel to the width direction of the grasping piece 12.

The substrate 41 has a substrate side surface (first substrate side surface) 46 facing one side in the width direction of the substrate 41 and a substrate side surface (second substrate side surface) 47 facing a side opposite to the substrate side surface 46. The substrate side surface 47 faces the other side in the width direction of the substrate 41. In a state where the heat-generating module 40 is attached to the installation surface 34 of the heat conduction member 32, the substrate side surface 46 faces one side of the end effector 5 in the width direction, and the substrate side surface 47 faces the other side of the end effector 5 in the width direction.

The substrate 41 includes a substrate distal surface 48 which forms a distal end of the substrate 41 and faces the distal side. The substrate distal surface 48 faces one side in a direction substantially perpendicular to the width direction and the thickness direction of the substrate 41. In addition, the substrate distal surface 48 is a surface which is substantially perpendicular to the substrate front surface 44, the substrate rear surface 45, and the substrate side surfaces 46 and 47. Further, the heat conduction member 32 includes a distal surface 38 which forms a distal end of the heat conduction member 32 and faces the distal side, and an inner wall surface 39 which faces the proximal side in a distal portion of the heat conduction member 32. The inner wall surface 39 is adjacent to the cavity 36 from the distal side inside the grasping piece 12. The inner wall surface 39 is located on the distal side from the substrate distal surface 48 and faces the substrate distal surface 48 of the substrate 41.

The heating element 42 is provided on the substrate front surface 44 of the substrate 41. The heating element 42 includes two connection terminals (not shown). The connection terminals are disposed in a proximal portion of the substrate front surface 44. One end of the electric supply path (not shown) formed of an electric wire or the like is connected to one of the connection terminals, and one end of another electric supply path (not shown) formed of an electric wire or the like is connected to the other of the connection terminals. Each of the electric supply paths extends through the inside of the shaft 2, the inside of the housing 3, and the inside of the cable 15, and each of the other end of the electric supply paths is connected to the energy source apparatus 17. In addition, the heating element 42 has a folding position (not shown). The folding position is located at the distal portion of the grasping piece 12. In the substrate front surface 44, the heating element 42 extends toward the distal side from the one connection terminal to the folding position, and the heating element 42 extends toward the proximal side from the folding position to the other connection terminal.

Based on the operation by the operation member 18, the energy source apparatus 17 outputs a direct current or an alternating current to the heating element 42 as electrical energy different from the electrical energy (high frequency energy) supplied to the conductive member 22 and the heat conduction member 32. A direct current or an alternating current flows to the heating element 42, and thus, heat is generated in the heating element 42. The heat generated by the heating element 42 is transmitted to the heat conduction member 32 via the adhesive layer 60 described later.

In the cavity 36, the adhesive layer 60 is provided between the heat-generating module 40 and the heat conduction member 32. The adhesive layer 60 extends over the range from the proximal portion of the grasping piece 12 to the distal portion thereof in the direction along the longitudinal axis C. The adhesive layer 60 adheres the heating element 42 and the substrate front surface 44 of the substrate 41 to the installation surface 34 of the heat conduction member 32. In addition, the adhesive layer 60 is formed of a material which has electrical insulation and a high thermal conductivity. For this reason, the heating element 42 and the installation surface 34 of the heat conduction member 32 is electrically insulated each other by the adhesive layer 60. For example, the adhesive layer 60 is formed of a mixture of an epoxy resin and ceramic.

The adhesive layer 60 has a front surface close contact portion 64 which is in close contact with the entire surface of the heating element 42 and the substrate front surface 44 of the substrate 41 from the treatment surface 33 side. In addition, the front surface close contact portion 64 is in close contact with the installation surface 34 of the heat conduction member 32 from the rear surface 35 side. The front surface close contact portion 64 adheres the substrate front surface 44 and the heating element 42 to the installation surface 34 of the heat conduction member 32 and electrically insulates between the heat-generating module 40 and the heat conduction member 32.

A boundary B is formed between the substrate front surface 44 and the front surface close contact portion 64. The boundary B is extended along the substrate front surface 44 of the substrate 41. The boundary B extends from the proximal portion of the substrate 41 to the distal portion thereof in the direction along the longitudinal axis C.

The adhesive layer 60 includes a rear surface close contact portion 65 which is in close contact with the substrate rear surface 45 of the substrate 41 from the rear surface 35 side. The substrate rear surface 45 is in close contact with the rear surface close contact portion 65, and thus, is not exposed to the cavity 36.

In addition, the adhesive layer 60 includes a side surface close contact portion 66 which is in close contact with the substrate side surface 46 of the substrate 41 from the outside in the width direction and a side surface close contact portion 67 which is in close contact with the substrate side surface 47 from the outside in the width direction. Each of the side surface close contact portions 66 and 67 is continued to the front surface close contact portion 64 and the rear surface close contact portion 65, respectively. The substrate side surfaces 46 and 47 and the boundary B are in close contact with the side surface close contact portions 66 and 67, and thus, are not exposed to the cavity 36.

The adhesive layer 60 includes a distal surface close contact portion 68 which is in close contact with the substrate distal surface 48 of the substrate 41 from the distal side. Moreover, the distal surface close contact portion 68 is continued to each of the front surface close contact portion 64, the side surface close contact portions 66 and 67, and the rear surface close contact portion 65. Accordingly, the substrate distal surface 48 and the boundary B are in close contact with the distal surface close contact portion 68, and thus, are not exposed to the cavity 36.

The front surface close contact portion 64 is continued to each of the side surface close contact portions 66 and 67. In addition, the rear surface close contact portion 65 is continued to each of the side surface close contact portions 66 and 67. Accordingly, the adhesive layer 60 is in close contact with the substrate 41 and the heating element 42 of the heat-generating module 40 from the outside over an entire periphery in a circumferential direction of the substrate 41. Therefore, in the present embodiment, an electrical insulation portion is formed by the adhesive layer 60, and the electrical insulation portion is formed of a material having electrical insulation and is in close contact with the heating element 42, the substrate front surface 44, the substrate rear surface 45, the substrate side surfaces 46 and 47, and the substrate distal surface 48.

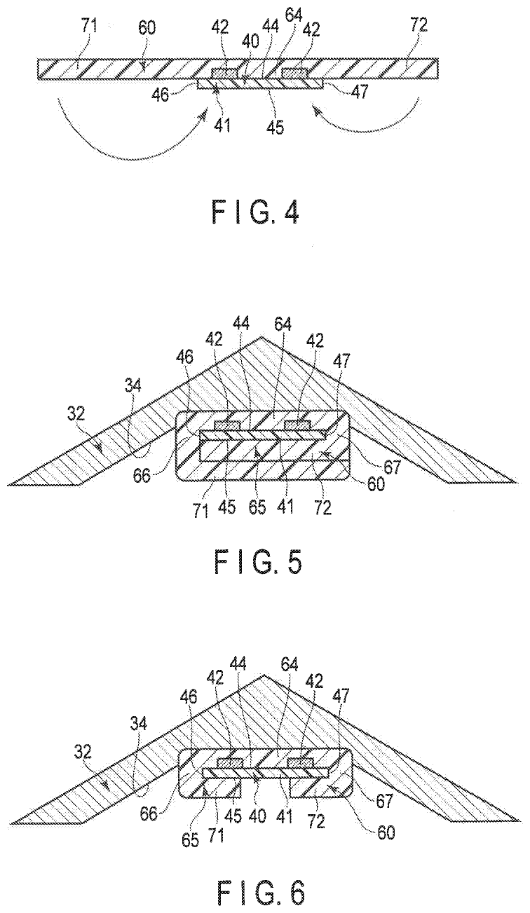

Next, a method of attaching the heat-generating module 40 to the heat conduction member 32 will be described with reference to FIGS. 4 and 5. In a manufacture of the treatment instrument 1, the heating elements 42 are formed on the substrate front surface 44 of the substrate 41, and the heat-generating module 40 including the substrate 41 and the heating elements 42 is adhered to the heat conduction member 32 via the adhesive layer 60.

For example, an adhesive sheet is used for the adhesive layer 60. In this case, first, the heat-generating module 40 is disposed in a state where the substrate front surface 44 including the heating elements 42 is in close contact with the adhesive layer 60 (adhesive sheet). An area of a surface in the adhesive layer (adhesive sheet) 60 on which the substrate 41 is disposed is sufficiently larger than an area of the substrate front surface 44 of the substrate 41. Therefore, the heat-generating module 40 is disposed in a state where the adhesive layer 60 is in close contact with the entire surface of the substrate front surface 44. In this case, in the adhesive layer 60, the front surface close contact portion 64 which is in close contact with the substrate front surface 44, a first extension portion 71 which extends outward from an edge of the substrate front surface 44 toward a side where the substrate side surface 46 faces in the width direction of the substrate 41, and a second extension portion 72 which extends from an edge of the substrate front surface 44 toward a side where the substrate side surface 47 faces in the width direction of the substrate 41 are formed.

Moreover, the adhesive layer 60 is folded so as to wrap the heat-generating module 40 in the circumferential direction of the substrate 41, and the adhesive layer 60 comes into close contact with each of the substrate side surfaces 46 and 47 and the substrate rear surface 45 of the substrate 41 from the outside. In this case, the second extension portion 72 comes into close contact with the substrate side surface 47 from the outside in the width direction and comes into close contact with the substrate rear surface 45 from the side to which the substrate rear surface 45 faces. The first extension portion 71 comes into close contact with the substrate side surface 46 from the outside in the width direction and comes into close contact with the substrate rear surface 45 and/or the second extension portion 72 from the side to which the substrate rear surface 45 faces. Therefore, the side surface close contact portion 67 and a portion of the rear surface close contact portion 65 are formed by the second extension portion 72, and the side surface close contact portion 66 and a portion of the rear surface close contact portion 65 are formed by the first extension portion 71.

In addition, in the substrate distal surface 48, a portion of the adhesive layer (adhesive sheet) 60 extending from the distal end of the substrate front surface 44 toward the distal side is folded, and thus, the adhesive layer 60 comes into close contact with the substrate distal surface 48 from the distal side, and the distal surface close contact portion 68 is formed. In the embodiment of FIGS. 4 and 5, in the state where the adhesive layer 60 is folded, the second extension portion 72 comes into close contact with the entire substrate rear surface 45, and the first extension portion 71 comes into close contact with the second extension portion 72 from the side where the substrate rear surface 45 faces. Moreover, the rear surface close contact portion 65 is formed by the first extension portion 71 and the second extension portion 72. In this case, the second extension portion 72 is disposed between the first extension portion 71 and the substrate rear surface 45, and the first extension portion 71 is exposed to the cavity 36. Accordingly, the substrate rear surface 45 is not exposed to the cavity 36 by the close contact of the adhesive layer 60.

In addition, in a state where the adhesive layer 60 is disposed around the heat-generating module 40 in the circumferential direction of the substrate 41, the adhesive layer 60 is heated, and thus, a temperature of the adhesive layer 60 increases to a predetermined temperature. Moreover, by using a press machine or the like, the substrate 41 is pressed against the installation surface 34 of the heat conduction member 32 so as to press the adhesive layer 60. In this case, a predetermined pressure is applied to the adhesive layer 60. As a result, the front surface close contact portion 64 of the adhesive layer 60 comes into close contact with the installation surface 34 from the side where the substrate 41 is located, and the front surface close contact portion 64 of the adhesive layer 60 is adhered to the installation surface 34 by a fluidized thermosetting resin or the like.

In addition, in a state where the adhesive layer 60 is in close contact with the substrate front surface 44 and the installation surface 34, heating of the adhesive layer 60 is continued. Accordingly, the thermosetting resin forming the adhesive layer 60 is chemically changed, and the adhesive layer 60 is cured. The adhesive layer 60 is cured, and thus, the substrate 41 (heat-generating module 40) is attached to the installation surface 34 of the heat conduction member 32 via the front surface close contact portion 64. Moreover, if the adhesive layer 60 is cured, the heating of the adhesive layer 60 is stopped.

In addition, it is also preferable that a thin film of aluminum or the like be disposed in a boundary portion formed between the first extension portion 71 and the second extension portion 72. In this case, adhesive properties between the first extension portion 71 and the second extension portion 72 are improved.

Next, an operation and effects of the treatment instrument 1 of the present embodiment will be described. When a treatment is performed using the treatment instrument 1, an operator holds the housing 3 of the treatment instrument 1 and inserts the end effector 5 into a body cavity such as an abdominal cavity. In addition, the treatment target such as a blood vessel is disposed between the grasping pieces 11 and 12, the handle 8 is closed with respect to the grip 7, and thus, the grasping pieces 11 and 12 is closed. Accordingly, the living tissue such as a blood vessel or the like is grasped between the grasping pieces 11 and 12 as the treatment target.

By inputting the operation by the operation member 18 in a state where the treatment target is grasped between the grasping pieces 11 and 12, the electrical energy is supplied from the energy source apparatus 17 to the heating elements 42. The electric energy is supplied to the heating elements 42, and thus, heat is generated in the heating elements 42. The heat generated by the heating elements 42 is transmitted to the heat conduction member 32 via the adhesive layer 60. The heat conduction member 32 is formed of a material having a high thermal conductivity. Accordingly, the heat transmitted from the heating elements 42 is transmitted to the entire heat conduction member 32. In addition, the heat transmitted to the heat conduction member 32 is applied to the treatment target from the treatment surface 33. As a result, the heat is applied to the treatment target grasped between the grasping pieces 11 and 12 as the treatment energy, and the treatment target is cut simultaneously with coagulation. In this way, in the treatment surface 33, the treatment for applying heat to the grasped treatment target is performed.

Further, in the present embodiment, the operation is input to the operation member 18, and thus, the electrical energy (high frequency electric power) is supplied from the energy source apparatus 17 to each of the heat conduction member 32 and the conductive member 22. By supplying the electrical energy to each of the heat conduction member 32 and the conductive member 22, the high frequency current flows to the portion between the treatment surface 33 and the conductive member 22 of the facing surface 23 through the treatment target grasped between the grasping pieces 11 and 12. Thereby, the high frequency current is applied to the treatment target between the treatment surface 33 and the facing surface 23. That is, the high frequency energy is supplied to the portion between the treatment surface 33 and the facing surface 23 as the treatment energy. The high frequency current is applied, and thus, the coagulation of the treatment target is promoted. Accordingly, in the treatment surface 33, high frequency energy (high frequency current) is supplied to the treatment target grasped between the grasping pieces 11 and 12.

In the present embodiment, the substrate side surfaces 46 and 47 are covered with the adhesive layer 60 by the close contact of the side surface close contact portions 66 and 67 of the adhesive layer 60. Accordingly, the substrate side surfaces 46 and 47 are not exposed to the cavity 36. In addition, the adhesive layer 60 is in close contact with the substrate side surfaces 46 and 47, and thus, the boundary B is not exposed to the cavity 36 and the installation surface 34 of the heat conduction member 32 on the substrate side surfaces 46 and 47. Therefore, the adhesive layer 60 having the electrical insulation is in close contact with the substrate side surfaces 46 and 47, and thus, in a state where the electrical energy is supplied to the heating element 42, discharge to the cavity 36 and the heat conduction member 32 (installation surface 34) through the gap of the boundary B between the substrate (substrate front surface 44) and the adhesive layer 60 (front surface close contact portion 64) is effectively prevented. Accordingly, electrical conduction between the heat conduction member 32 and the heating element 42 is effectively prevented, and a voltage resistance of an electrical insulation site formed from the adhesive layer 60 is improved.

In addition, in the present embodiment, the distal surface close contact portion 68 of the adhesive layer 60 having electrical insulation is in close contact with the substrate distal surface 48. Accordingly, the substrate distal surface 48 is not exposed to the cavity 36. In addition, the boundary B is not exposed to the cavity 36 and the inner wall surface 39 of the heat conduction member 32 in the substrate distal surface 48. Therefore, the adhesive layer 60 having the electrical insulation is in close contact with the substrate distal surface 48, and thus, in the state where the electrical energy is supplied to the heating element 42, discharge to the cavity 36 and the heat conduction member 32 (inner wall surface 39) through the gap of the boundary B between the substrate 41 (substrate front surface 44) and the adhesive layer 60 (front surface close contact portion 64) is effectively prevented. Accordingly, electrical conduction between the heat conduction member 32 and the heating element 42 is effectively prevented, and the voltage resistance of an electrical insulation site formed from the adhesive layer 60 is improved.

FIG. 6 is a view showing an example (manufacturing example) in a state where an adhesive layer (adhesive sheet) 60 is disposed around a substrate 41. In a state where the adhesive layer 60 is folded, each of a first extension portion 71 and a second extension portion 72 comes into close contact with a portion of a substrate rear surface 45 in a state where the first extension portion 71 and the second extension portion 72 do not come into contact with each other. In this case, a portion of the substrate rear surface 45 is exposed to the cavity 36. Moreover, a rear surface close contact portion 65 which is in close contact with the substrate rear surface 45 may not be provided.

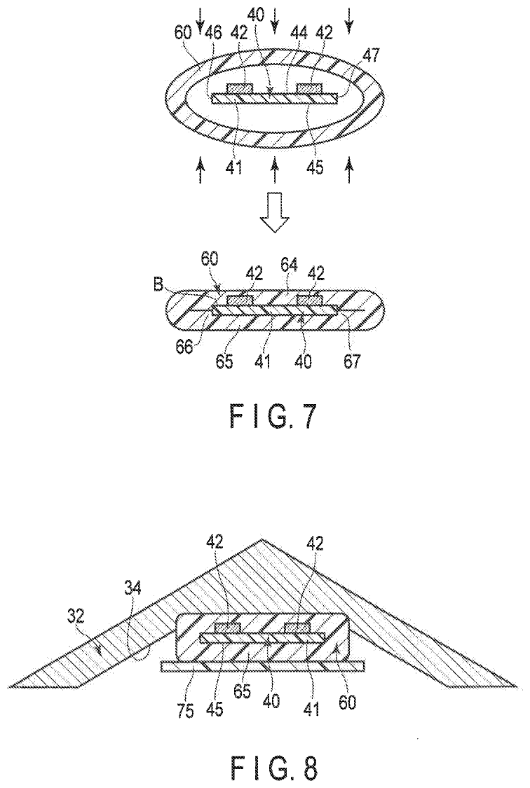

In an example (manufacturing example) of FIG. 7, an adhesive sheet formed in a tube shape is used in the adhesive layer 60. In this case, a heat-generating module 40 is disposed in a cavity of an adhesive layer (tube) 60 formed in a tube shape. In this case, the adhesive layer 60 is disposed over an entire periphery in a circumferential direction of a substrate 41 around the heat-generating module 40. In this state, as described above, if the adhesive layer 60 is heated and pressured, the adhesive layer (tube) 60 is shrunk toward the substrate 41. Accordingly, the adhesive layer 60 comes into close contact with the heat-generating module 40 from the outside over the entire periphery in the circumferential direction of the substrate 41. In addition, a front surface close contact portion 64, side surface close contact portions 66 and 67, and a rear surface close contact portion 65 are formed. Moreover, by the tube for the adhesive layer 60, assembly is easily performed when the adhesive layer 60 is formed, and a configuration can be easily formed, in which a boundary B between the substrate 41 and the adhesive layer 60 is not exposed to a cavity 36 in substrate side surfaces 46 and 47. Accordingly, it is easy to guarantee (secure) the voltage resistance of the electrical insulation site formed by the adhesive layer 60.

FIG. 8 is a view showing configurations of a heat conduction member 32 and a heat-generating module 40 in a third modification of the present embodiment. As shown in FIG. 8, in the present modification, a protective layer 75 is provided on a substrate rear surface 45 of a substrate 41 of the heat-generating module 40 via a rear surface close contact portion 65 of an adhesive layer 60. The protective layer 75 comes into close contact with the rear surface close contact portion 65 from a rear surface 35 side. The protective layer 75 is formed of a material having heat resistance and water resistance. For example, the protective layer 75 is a film-shaped thin film formed of a highly functional resin such as PEEK (polyetheretherketone) or mica. In addition, it is preferable that the protective layer 75 be a coating formed of an air layer coating material such as parylene. Further, it is also preferable that a plurality of protective layers 75 formed to be laminated on the rear surface 35 side be provided on the substrate rear surface 45.

In the present modification, the rear surface close contact portion 65 is prevented from being exposed to the cavity 36 by the protective layer 75 in close contact with the rear surface close contact portion 65 of the adhesive layer 60. Therefore, in the cavity 36, air, moisture, and other substances are prevented from coming into contact with the adhesive layer 60 from the rear surface 35 side by the protective layer 75. By preventing air from coming into contact with the adhesive layer 60, oxidation and deterioration of the adhesive layer 60 are prevented. In addition, by preventing water from coming into contact with the adhesive layer 60, the water resistance of the adhesive layer 60 is improved. In this way, by providing the protective layer 75 in the adhesive layer 60, influences of the air, moisture, and other substances on the adhesive layer 60 are prevented.

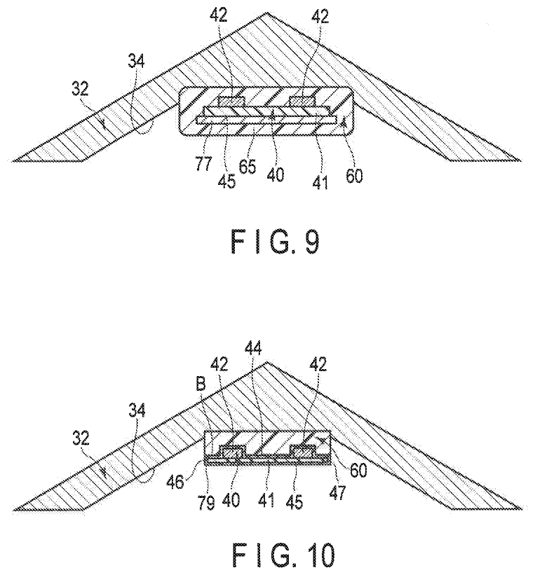

FIG. 9 is a view showing configurations of a heat conduction member 32 and a heat-generating module 40 in a fourth modification of the present embodiment. As shown in FIG. 9, in the present modification, an anisotropic member (anisotropic material) 77 is disposed on a substrate rear surface 45 of a substrate 41 of the heat-generating module 40. The anisotropic member 77 is disposed between the substrate rear surface 45 and a rear surface close contact portion 65 of an adhesive layer 60. The anisotropic member 77 is in close contact with the substrate rear surface 45 from a rear surface 35 side.

The anisotropic member 77 is formed in a plate shape and is formed of a material (element) having anisotropy of heat conduction. The anisotropic member 77 has a property that it is easy to transmit heat in a plane direction, but it is difficult to transmit heat in a thickness direction. For example, graphite is used for the anisotropic member 77. A thermal conductivity in the plane direction of the graphite is about 5000 to 6000 W/m·K, and a thermal conductivity in the thickness direction is about 5 to 20 W/m·K. The anisotropic member 77 is attached to the substrate rear surface 45 of the substrate 41 in a state where a plate surface of the anisotropic member faces the substrate rear surface 45. Therefore, in the anisotropic member 77, a thermal conductivity in the plane direction, that is, a direction along the substrate rear surface 45 (direction along a width direction of the substrate 41 and a longitudinal axis C) is higher than a thermal conductivity in the thickness direction, that is, a direction in which the substrate rear surface 45 faces. In addition, the thermal conductivity of the anisotropic member 77 in the plane direction is higher than a thermal conductivity of the heat conduction member 32, and the thermal conductivity of the anisotropic member 77 in the thickness direction is lower than the thermal conductivity of the heat conduction member 32. In addition, the anisotropic member 77 may be formed of a plurality of laminated graphite.

In the present modification, heat transmitted from heating elements 42 to the anisotropic member 77 via the substrate 41 is transmitted to the entire anisotropic member 77 in the width direction of the substrate 41 and the direction along the longitudinal axis C. Moreover, the heat transmitted to the entire anisotropic member 77 is transmitted to the heat conduction member 32 via the substrate 41. Accordingly, the heat generated by the heating elements 42 is transmitted to a treatment surface 33 of the heat conduction member 32 uniformly in the direction along the longitudinal axis C. The heat is uniformly transmitted to the heat conduction member 32, and thus, the heat is prevented from being concentrated on a portion of the treatment surface 33.

In addition, the anisotropic member 77 hardly transmits heat from the substrate rear surface 45 to the rear surface 35 side. Therefore, the heat generated by the heating elements 42 is prevented from being transmitted via the cavity 36 to another member (for example, the support 31) disposed on the rear surface 35 side of the heat-generating module 40 inside a grasping piece 12. Accordingly, the heat is prevented from entering another member disposed on the rear surface 35 side of the heat-generating module 40. In addition, the graphite is more easily oxidized than the material (element) used for the adhesive layer 60. Accordingly, in a case where the graphite is used for the anisotropic member 77, the graphite is oxidized before the adhesive layer 60, and thus, the oxidation of the adhesive layer 60 is prevented.

FIG. 10 is a view showing configurations of a heat conduction member 32 and a heat-generating module 40 in a fifth modification of the present embodiment. As shown in FIG. 10, in the present modification, an adhesive layer 60 is provided only between an installation surface 34 of the heat conduction member 32 and a substrate front surface 44. Further, in the heat-generating module 40, an insulating portion 79 is formed by coating over an entire periphery in a circumferential direction of a substrate 41. The adhesive layer 60 is disposed between the installation surface 34 of the heat conduction member 32 and the insulating portion 79. The insulating portion 79 is a thin film formed by a coating material having electrical insulation. A ceramic coating, parylene, or the like is used as the coating material for forming the insulating portion 79, and it is preferable that an air-layer coating material such as parylene be used.

The adhesive layer 60 is in close contact with the installation surface 34 of the heat conduction member 32 from the rear surface 35 side between the installation surface 34 of the heat conduction member 32 and the substrate front surface 44, and the insulating portion 79 is in close contact with the adhesive layer 60 from the rear surface 35 side. In addition, the insulating portion 79 is in close contact with the substrate front surface 44 from a treatment surface 33 side. In the present modification, the adhesive layer 60 may be formed of a material (conductor, semiconductor, or the like) which does not have electrical insulation.

In substrate side surfaces 46 and 47, the insulating portion 79 is exposed to the cavity 36. Accordingly, the substrate side surfaces 46 and 47 are not exposed to the cavity 36 by the close contact of the insulating portion 79. In the present modification, the insulating portion 79 exists between the heating elements 42 and the substrate front surface 44, and the adhesive layer 60. Accordingly, discharge from the heating elements 42 to a boundary B between the substrate 41 having the insulating portion 79 and the adhesive layer 60 is prevented. Accordingly, discharge from the heating elements 42 to the installation surface 34 of the heat conduction member 32 via the boundary B is prevented.

Moreover, the insulating portion 79 having electrical insulation is in close contact with the substrate side surfaces 46 and 47. Accordingly, discharge from the substrate side surfaces 46 and 47 to the installation surface 34 between the heating element 42 and the heat conduction member 32 is prevented.

In addition, the insulating portion 79 having electrical insulation is in close contact with a substrate distal surface 48. Accordingly, discharge from the substrate distal surface 48 to an inner wall surface 39 between the heating element 42 and the heat conduction member 32 is prevented.

In addition, in the present modification, coating of the insulating portion 79 provided between the substrate front surface 44 and the adhesive layer 60 improves adhesive properties between the substrate front surface 44 and the adhesive layer 60. Further, the close contact of the insulating portion 79 to the substrate rear surface 45 improves water tightness and prevents water or the like from coming into contact with the heat-generating module 40.

Moreover, in the above-described embodiment or the like, a bipolar treatment is performed, in which the heat conduction member 32 and the conductive member 22 function as the electrodes and the high frequency current flows to the portion between the heat conduction member 32 and the conductive member 22 through a treatment target. However, the present invention is not limited to this. For example, in a modification, a grasping piece 11 may not be provided, and a treating portion having a configuration similar to that of a grasping piece 12 may be provided on a distal portion of a shaft 2. In this case, a system in which a treatment instrument 1 is used is provided with a counter electrode plate (not shown), and in the treatment, the counter electrode plate is attached to a human body or the like outside a body. In the present modification, high frequency electric power is supplied from an energy source apparatus 17 to the heat conduction member 32 and the counter electrode plate. In addition, a monopolar treatment is performed, in which high frequency current flows through the treatment target between a treatment surface 33 of the heat conduction member 32 and the counter electrode plate. In addition, in the present modification, electric energy (direct current electric power or alternating current electric power) is supplied to heating elements 42, and thus, heat is generated in the heating elements 42. In addition, the heat generated by the heating elements 42 is transmitted to the treatment surface 33 through an adhesive layer 60 and the heat conduction member 32 and is applied to the treatment target from the treatment surface 33.

In the above-described embodiment or the like, a treatment instrument (1) includes: a heat conduction member (32) which includes a treatment surface (33) and an installation surface (34) facing a side opposite to the treatment surface (33), has a thermal conductivity, and functions as an electrode with electrical energy supplied; a heating element (42) which generates heat with electrical energy supplied; a substrate (41) which comprises a substrate front surface (44) on which the heating element (42) is formed and a substrate side surface (46, 47) facing a width direction, and is attached to the installation surface (34) of the heat conduction member (32) in a state where the substrate front surface (44) faces a side on which the heat conduction member (32) is located; an adhesive layer (60) which is provided between the installation surface (34) of the heat conduction member (32) and the substrate (41), is formed of a material having a thermal conductivity, and is in close contact with the installation surface (34) of the heat conduction member (32); and an insulating portion (60; 79) which is formed of a material having electrical insulation and is in close contact with the heating element (42), the substrate front surface (44), and the substrate side surface (46, 47).

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A treatment instrument comprising:
a heat conductor that includes a treatment surface and an installation surface on a side opposite to the treatment surface;
a heat generator that generates heat when electrical energy is supplied to the heat generator;
a substrate that includes:
a substrate front surface, the heat generator being formed on the substrate front surface,
the substrate front surface being configured to face the heat conductor; an adhesive layer that is provided between the installation surface of the heat conductor and the substrate, the adhesive layer being formed of a material having a thermal conductivity and contacting the installation surface of the heat conductor; and
an insulating portion formed of a material having electrical insulation, the insulating portion contacting the heat generator and the substrate.

2. The treatment instrument according to claim 1, the substrate further comprising a first substrate side surface on a side of the substrate and a second substrate side surface on an opposite side of the substrate, wherein: the insulating portion contacts the first substrate side surface and the second substrate side surface.

3. The treatment instrument according to claim 1,
wherein the insulating portion is formed by the adhesive layer.

4. The treatment instrument according to claim 1, further comprising:
a grasping piece that faces the treatment surface of the heat conductor and is configured to open and close relative to the heat conductor, wherein:
the heat conductor has an electrical conductivity and functions as an electrode when electrical energy is supplied to the heat conductor, and
the grasping piece includes an electrical conductor, the electrical conductor functioning as an electrode different from the heat conductor when electrical energy is supplied to the conductor.

5. The treatment instrument according to claim 1,
wherein the substrate includes a substrate rear surface provided on a side opposite the substrate front surface, and
the insulating portion contacts at least a portion of the substrate rear surface.

6. The treatment instrument according to claim 5,
wherein the insulating portion contacts an entire periphery of the substrate.

7. The treatment instrument according to claim 1, wherein:
the substrate includes a substrate distal surface on a distal side of the treatment instrument, and
the insulating portion contacts the substrate distal surface.

8. The treatment instrument according to claim 1, wherein:
the insulating portion is a coating that is water resistant; and
the adhesive layer contacts the insulating portion on a side on which the heat conductor is located.

9. The treatment instrument according to claim 1,
wherein the substrate includes a substrate rear surface provided on a side opposite the substrate front surface, and
the treatment instrument further comprises a protective layer provided on the substrate rear surface, the protecting layer being water resistant.

10. The treatment instrument according to claim 1,
wherein the substrate includes a substrate rear surface provided on a side opposite the substrate front surface, and
the treatment instrument further comprises an anisotropic material provided on the substrate rear surface and has thermal conductivity anisotropy.

11. The treatment instrument according to claim 10,
wherein the anisotropic material has a higher thermal conductivity in a direction along the substrate rear surface than a thermal conductivity in a direction in which the substrate rear surface faces.

12. The treatment instrument according to claim 10,
wherein the anisotropic material includes a material which is more easily oxidized than the adhesive layer.

13. The treatment instrument according to claim 10,
wherein the anisotropic material is graphite.

14. A treatment instrument comprising:
a heat conductor that has thermal conductivity and includes a treatment surface and an installation surface provided on a side opposite the treatment surface;
a heat generator that generates heat when electrical energy is supplied to the heat generator;
a substrate that includes a substrate front surface, the heat generator being formed on the substrate front surface, the substrate being attached to the installation surface of the heat conductor such that the substrate front surface faces the heat conductor;
an adhesive layer that is provided between the installation surface of the heat conductor and the substrate, the adhesive layer being formed of a material having a thermal conductivity and contacting the installation surface of the heat conductor; and
an insulating portion that is formed of a material having electrical insulation, the insulating portion contacting the heat generator, the substrate front surface,
wherein the insulating portion is formed by the adhesive layer.
